SEQUENCE LISTING (7 sequences)

SEQ ID NO: 1 - DNA, 20 bp, Artificial Sequence
  Description of Artificial Sequence Synthetic oligonucleotide
hbdvhbdvhbdvhbdvhbdv

SEQ ID NO: 2 - DNA, 20 bp, Artificial Sequence
  Description of Artificial Sequence Synthetic oligonucleotide
vdbhvdbhvdbhvdbhvdbh

SEQ ID NO: 3 - DNA, 27 bp, Artificial Sequence
  Description of Artificial Sequence Synthetic oligonucleotide
gtaggtgtgagtgatggttgaggtagt

SEQ ID NO: 4 - DNA, 27 bp, Artificial Sequence
  Description of Artificial Sequence Synthetic oligonucleotide
gtgagtgatggttgaggtagtgtggag

SEQ ID NO: 5 - DNA, 19 bp, Artificial Sequence
  Description of Artificial Sequence Synthetic oligonucleotide
agtcgcttgggtgtagtgc

SEQ ID NO: 6 - DNA, 30 bp, Artificial Sequence
  Description of Artificial Sequence Synthetic oligonucleotide
  misc_feature (1)..(30): This sequence may encompass 10-30 nucleotides
tttttttttttttttttttttttttttttt

SEQ ID NO: 7 - DNA, 12 bp, Artificial Sequence
  Description of Artificial Sequence Synthetic oligonucleotide
tttttttttttt

(19) United States
(12) Patent Application Publication
(10) Pub. No.: US 2020/0181606 A1
(43) Pub. Date: Jun. 11, 2020

(54) A Method of Amplifying Single Cell Transcriptome

(71) Applicant: President and Fellows of Harvard College, Cambridge, MA (US)

(72) Inventors: Xiaoliang Sunney Xie, Lexington, MA (US); Alec R. Chapman, Cambridge, MA (US); David F. Lee, Somerville, MA (US)

(21) Appl. No.: 16/617,643

(22) PCT Filed: May 25, 2018
(86) PCT No.: PCT/US18/34689

§ 371 Date: Nov. 27, 2019

Related U.S. Application Data
(60) Provisional application No. 62/512,144, filed on May 29, 2017.

(51) Int. Cl. C12N 15/10

(52) U.S. Cl. CPC C12N 15/1096

(57) ABSTRACT

The present disclosure provides a method for amplifying RNA using a combination of reverse transcription and multiple annealing and looping based amplification cycles. Primers are used such that the resulting amplicons include a first cell specific barcode sequence, a second cell specific barcode sequence and a unique molecular identifier barcode sequence.

### RELATED APPLICATION DATA

This application claims priority to U.S. Provisional Application No. 62/512,144 filed on May 29, 2017, which is hereby incorporated herein by reference in its entirety for all purposes.

### STATEMENT OF GOVERNMENT INTERESTS

This invention was made with government support under CA174560 and CA186693 from the National Institutes of Health. The Government has certain rights in the invention.

### BACKGROUND

### Field of the Invention

Embodiments of the present invention relate in general to methods and compositions for single cell messenger RNA amplification, such as messenger RNA from a single cell.

### Description of Related Art

Single cell RNA sequencing technologies are known. See Wen et al., Genome Biology (2016) 17:17, DOI 10.1186/s13059-016-0941-0; Mortazavi et al., Nature Methods DOI: 10.1038/nmeth.1226; Chapman et al., PLoS ONE 10(3): e0120889, doi:10.1371/journal.pone.0120889 (2015); and Sheng et al., Nature Methods DOI: 10.1038/NMETH.4145 (2017). The first report of scRNA-seq by Tang et. al et al. (2009) mRNA-Seq whole-transcriptome analysis of a single cell. Nat Methods, 6, 377-382 used a poly-T primer for cDNA synthesis, followed by poly-A tailing, second strand synthesis and PCR. Subsequent technological advancements include the addition of template switching to improve RNA recovery efficiency (see Islam. S., Kjallquist, U., Moliner. A., Zajac, P., Fan, J. B., Lonnerberg, P. and Linnarsson, S. (2011) Characterization of the single-cell transcriptional landscape by highly multiplex RNA-seq. Genome Res, 21, 1160-1167; Picelli, S., Bjorklund. A. K., Faridani, O. R., Sagasser, S., Winberg. G. and Sandberg, R. (2013) Smart-seq2 for sensitive full-length transcriptome profiling in single cells. Nat Methods, 10, 1096-109), cell-specific barcodes to allow sample multiplexing (see Jaitin, D. A., Kenigsberg. E., Keren-Shaul, H., Elefant, N., Paul, F., Zaretsky, I., Mildner, A., Cohen, N., Jung. S., Tanay, A. et al. (2014) Massively parallel single-cell RNA-seq for marker-free decomposition of tissues into cell types. Science, 343, 776-779; Fan. H. C., Fu, G. K. and Fodor, S. P. (2015) Expression profiling. Combinatorial labeling of single cells for gene expression cytometry. Science, 347, 1258367), optimized enzymatic conditions (see Sasagawa, Y., Nikaido, I., Hayashi, T., Danno, H., Uno, K. D., Imai, T. and Ueda. H. R. (2013) Quartz-Seq: a highly reproducible and sensitive single-cell RNA sequencing method, reveals non-genetic gene-expression heterogeneity. Genome Biol, 14. R31), unique molecular identifiers to tag unique cDNAs (see Islam. S., Zeisel, A., Joost, S., La Manno, G., Zajac, P., Kasper, M., Lonnerberg, P. and Linnarsson, S. (2014) Quantitative single-cell RNA-seq with unique molecular identifiers. Nat Methods, 11, 163-166; Shiroguchi, K., Jia, T. Z., Sims. P. A. and Xie, X. S. (2012) Digital RNA sequencing minimizes sequence-dependent bias and amplification noise with optimized single-molecule barcodes. Proc Natl Acad Sci USA. 109, 1347-1352), in vitro transcription of cDNA to reduce amplification bias (see Hashimshony, T., Senderovich, N., Avital, G., Klochendler. A., de Leeuw, Y., Anavy, L., Gennert. D., Li, S., Livak, K. J., Rozenblatt-Rosen. O. et al. (2016) CEL-Seq2: sensitive highly-multiplexed single-cell RNA-Seq. Genome Biol, 17, 77), AND automation using microfluidic devices (Zheng, G. X., Terry. J. M., Belgrader, P., Ryvkin, P., Bent. Z. W., Wilson, R., Ziraldo, S. B., Wheeler, T. D., McDermott, G. P., Zhu. J. et al. (2017) Massively parallel digital transcriptional profiling of single cells. Nat Commun, 8, 14049, Macosko. E. Z., Basu. A., Satija, R., Nemesh, J., Shekhar, K., Goldman, M., Tirosh. I., Bialas, A. R., Kamitaki, N., Martersteck. E. M. et al. (2015) Highly Parallel Genome-wide Expression Profiling of Individual Cells Using Nanoliter Droplets. Cell, 161, 1202-1214; Klein. A. M., Mazutis, L., Akartuna, 1., Tallapragada, N., Veres, A., Li, V., Peshkin, L., Weitz, D. A. and Kirschner. M. W. (2015) Droplet barcoding for single-cell transcriptomics applied to embryonic stem cells. Cell, 161, 1187-1201).

Despite these advancements, one common limitation of these methods is low RNA detection efficiency, which is typically 20% or lower (see Ziegenhain. C., Vieth. B., Parekh, S., Reinius, B., Guillaumet-Adkins, A., Smets, M., Leonhardt, H., Heyn, H., Hellmann, I. and Enard. W. (2017) Comparative Analysis of Single-Cell RNA Sequencing Methods. Mol Cell, 65, 631-643 e634; Liu. S. and Trapnell, C. (2016) Single-cell transcriptome sequencing: recent advances and remaining challenges. F1000Res, 5). This adds uncertainty to RNA quantification due to sampling noise and causes dropout of lowly expressed transcripts. Another limitation is that, despite the addition of UMIs, RNA quantification is still inaccurate due to UMI miscounting. This occurs because UMI-containing reverse transcription primers may not be completely removed prior to cDNA amplification, and existing methods have no way to measure removal efficiency. Finally, for methods that use PCR to amplify cDNA, the exponential amplification process can cause amplification bias. Overall, these problems limit the completeness, accuracy, and cost-effectiveness of existing scRNA-seq methods. Accordingly, a need exists for further methods of amplifying small amounts of RNA, such as from a single cell or a small group of cells, which do not suffer from one or more drawbacks.

### SUMMARY

Embodiments of the present disclosure are directed to a method of amplifying RNA such as a small amount of RNA or a limited amount of RNA such as a RNA obtained from a single cell or a plurality of cells of the same cell type or from a tissue, fluid or blood sample obtained from an individual or a substrate. The methods described herein include reverse transcribing the RNA using primers as described to generate cDNA and then amplifying the cDNA according to multiple annealing and looping based amplification cycles described herein (see Method of amplifying genomic DNA from a single cell is described in Zong, C., Lu. S., Chapman, A. R., and Xie, X. S. (2012), Genome-wide detection of single-nucleotide and copy-number variations of a single human cell, Science 338, 1622-1626 which describes Multiple Annealing and Looping-Based Amplification Cycles (MALBAC) hereby incorporated by reference in its entirety) to produce double stranded amplicons having a first cell specific barcode, a second cell specific barcode and a unique molecular identifier barcode sequence as described herein. According to certain aspects of the present disclosure, the methods described herein can be performed in a single tube with programmable thermocycles.

The method described herein for single-cell RNA amplification may be referred to as Multiple Annealing and Looping Based Amplification Cycles for Digital Transcriptomics (MALBAC-DT) which overcomes drawbacks with other methods. The MALBAC-DT method described herein has higher RNA detection efficiency due to the use of random primers to anneal cDNA during cDNA amplification, which improves capture efficiency. Furthermore, the quasilinear cDNA amplification reduces amplification bias and hence transcript dropout. In addition, the MALBAC-DT method described herein has higher accuracy due to the UMI design. One aspect further includes a method to measure the efficiency of reverse transcription primer degradation before cDNA amplification.

According to one aspect, reverse transcription primers are used that include a 3′ poly(T) sequence complementary to a 5′ poly(A) sequence of an RNA template strand. The reverse transcriptase primer further includes a 5′ self-annealing sequence, a barcode primer annealing site, a first cell specific barcode sequence and a first unique molecular identifier barcode sequence to produce a cDNA corresponding to the RNA template, wherein the cDNA also includes the reverse transcription primer.

The cDNA is then subjected at a first low temperature to primers having the self-annealing sequence at the 5′ end of the primer, wherein the complementary strand includes the self-annealing sequence at the 5′ end and its complement at the 3′ end, where the primers anneal to the cDNA. Primer extension at a higher temperature then follows in the presence of at least one polymerase, such as a strand displacing polymerase or polymerases with 5′ to 3′ exonuclease activity. The extension product and the cDNA template are separated and then the mixture is subject to a lower temperature at which ends of the extension product anneal to themselves to form a loop thereby making the extension product unavailable for further extension or amplification. The cDNA template is then again extended in the manner above followed by looping of the extension product. The process is repeated a plurality of time to provide a population of looped extension products. The looped extension products are then dehybridized or melted and the single strands are then amplified using primers which include a second cell specific barcode sequence. The amplification results in double stranded amplicons including a first cell specific barcode sequence, a second cell specific barcode sequence and a unique molecular identifier sequence (UMI) where the UMI has a semi-random sequence. According to one aspect, several thermocycles take place to amplify the cDNA and form looped extension products that inhibit the extension product from being further extended or amplified. The amplification may be referred to as linear amplification or quasi-linear amplification. The looped extension products may then be amplified using standard or non-standard PCR cycles. Certain polymerases provide exemplary results.

According to certain aspects, methods are provided for processing at least one cell, one or more cells, or a plurality of cells, such as two or more cells for example for RNA amplification according to the methods described herein. According to an exemplary embodiment, a single cell is isolated and then lysed in a volume of fluid to obtain the RNA of the cell. According to an exemplary embodiment, multiple single cells may each be isolated and then lysed in a volume of fluid to obtain the RNA of the cell and then the RNA of the cells may be multiplex reverse transcribed and amplified.

Further features and advantages of certain embodiments of the present disclosure will become more fully apparent in the following description of the embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present invention will be more fully understood from the following detailed description of illustrative embodiments taken in conjunction with the accompanying drawings in which:

FIG. 1 depicts in schematic a method of making cDNA from mRNA transcript. A poly(T) containing primer (RT-Aₙ) with UMI pattern ‘A’ (UMI_{A}) and cell barcode Cₙ is annealed to the poly(A) region of the target mRNAs. Incubation with SuperScript IV, a reverse transcriptase, catalyzes cDNA synthesis. Exonuclease I is then added to digest any remaining RT primers and prevent them from priming during cDNA amplification. Addition of primer RT-Bₙ, which has the UMI_{B} pattern instead of the UMI_{A} pattern, allows the efficiency of exonuclease degradation to be measured since incomplete digestion will result in a mixture of UMI_{A} and UMI_{B} cDNA amplification products. Finally, the mix is incubated at 80° C. to degrade the RNA and heat inactivate Exonuclease I and Superscript IV.

FIG. 2 depicts in schematic a method of amplifying cDNA using multiple annealing and looping based amplification cycles (MALBAC). A primer (GAT5-7N) containing the GAT5 sequence and a 7-nucleotide random sequence anneals randomly to the cDNA. The primer may also contain the B1 spacer sequence. Incubation with 3′->5′ exonuclease deficient Deep Vent, a DNA polymerase, catalyzes second strand synthesis. Denaturation of these strands followed by cooling causes the second strand to form a stable hairpin loop structure, preventing further amplification. This is repeated 9 times to generate multiple loops and amplify the cDNA in a quasilinear fashion. After these quasilinear steps, the loops are denatured and amplified by PCR for 17 cycles using the GAT5-B1 primer. Finally, following MALBAC, the outer barcode primer is added and another 5 cycles of PCR performed with outer barcode and GAT5-B1 primers.

FIG. 3 depicts in schematic a library preparation protocol using a transposon based method called tagmentation. Tagmentation using a hyperactive Tn5 transposase, such as from the Nextera DNA Library Preparation Kit, produces multiple products, with the desired product having the barcode sequences and Read1SP flanking the cDNA. After gap repair at 72° C. with a DNA Polymerase, the Illumina sequencing compatible library is produced by 5 cycles of PCR using the Read 1 index adapter primer (called SSXX by Illumina) and the read 2 index adapter primer. Index1/Index2 are the Illumina sequencing indexes, and P5/P7 are the flowcell annealing adapters.

FIG. 4A depicts data of a correlation matrix for mRNAS of 12,000 consistently detected genes within ˜700 sequenced cells for a HEK293T culture (upper). FIG. 4B depicts clustering of genes (left) and FIG. 4C depicts clustering of cells (right) for the HEK293T dataset using the t-stochastic neighbor embedding algorithm (t-SNE). In the gene clustering plot of FIG. 4B, each gene cluster corresponds to a square in the correlation matrix. In the gene clustering plot, each dot is one of the 12,000 genes and each cluster corresponds to a square in the correlation matrix. In the cell clustering plot of FIG. 4C, each dot is one of ˜700 HEK cells, and there are no resolvable clusters.

FIG. 5 depicts data of a correlation matrix for mRNAs for 3000 out of 12,000 consistently detected genes within a HEK293T culture (upper). FIG. 5 depicts data of a correlation matrix for mRNAs for 3000 out of 12,000 consistently detected genes within a U-2 OS culture (lower). The color intensities are related to the Pearson correlation coefficient between two genes. Each square block on the diagonal indicates a gene cluster in which strong correlation is observed. The gene clusters are groups of genes which likely have common transcriptional regulation and biological function. Two of the cell clusters which are shared between the two cell lines are labeled as the cell cycle and protein synthesis clusters.

FIG. 6 highlights the protein synthesis cluster labeled in FIG. 5. Genes in this cluster are enriched for those involved in tRNA synthesis, amino acid synthesis, amino acid transport, and control of translation initiation, all of which are important in the protein synthesis process. Therefore, correlated gene clusters have related biological functions and transcriptional regulation.

FIG. 7 compares correlated modules between U-2 OS and HEK293T cell lines. Some modules related to universal cell functions such as cell cycle progression and protein synthesis are common to both cell lines, but others such as the p53 and bone extracellular matrix modules are specific to one cell type. This cell-type specificity is not necessarily reflected in differential expression. Some modules are still preserved despite differential expression between the two cell lines, while other modules disappear despite not being differentially expressed.

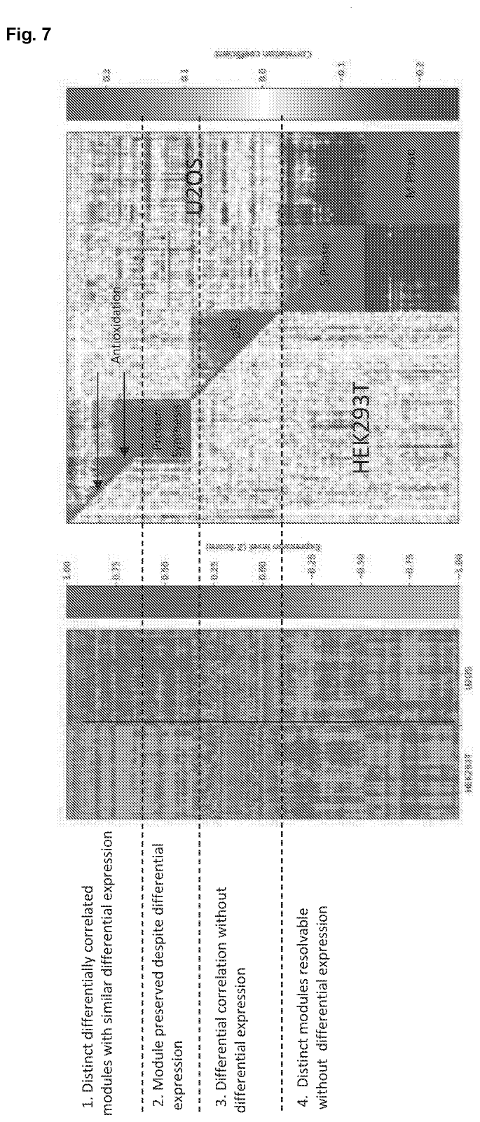

### DETAILED DESCRIPTION

The practice of certain embodiments or features of certain embodiments may employ, unless otherwise indicated, conventional techniques of molecular biology, microbiology, recombinant DNA, and so forth which are within ordinary skill in the art. Such techniques are explained fully in the literature. See e.g., Sambrook. Fritsch, and Maniatis. MOLECULAR CLONING: A LABORATORY MANUAL, Second Edition (1989), OLIGONUCLEOTIDE SYNTHESIS (M. J. Gait Ed., 1984), ANIMAL CELL CULTURE (R. I. Freshney, Ed., 1987), the series METHODS IN ENZYMOLOGY (Academic Press, Inc.); GENE TRANSFER VECTORS FOR MAMMALIAN CELLS (J. M. Miller and M. P. Calos eds. 1987), HANDBOOK OF EXPERIMENTAL IMMUNOLOGY, (D. M. Weir and C. C. Blackwell, Eds.), CURRENT PROTOCOLS IN MOLECULAR BIOLOGY (F. M. Ausubel, R. Brent, R. E. Kingston, D. D. Moore, J. G. Siedman, J. A. Smith, and K. Struhl, eds., 1987), CURRENT PROTOCOLS IN IMMUNOLOGY (J. E. coligan, A. M. Kruisbeek, D. H. Margulies, E. M. Shevach and W. Strober, eds., 1991); ANNUAL REVIEW OF IMMUNOLOGY; as well as monographs in journals such as ADVANCES IN IMMUNOLOGY. All patents, patent applications, and publications mentioned herein, both supra and infra, are hereby incorporated herein by reference.

Terms and symbols of nucleic acid chemistry, biochemistry, genetics, and molecular biology used herein follow those of standard treatises and texts in the field, e.g., Kornherg and Baker, DNA Replication, Second Edition (W.H. Freeman, New York, 1992); Lehninger, Biochemistry, Second Edition (Worth Publishers, New York, 1975); Strachan and Read, Human Molecular Genetics. Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

The present invention is based in part on the discovery of methods of amplifying one or more or a plurality of target RNA sequences from a cell or collection of cells, where the resulting amplicons include a first cell specific barcode sequence, a second cell specific barcode sequence and a unique molecular identifier barcode sequence. The amplicons can be processed into a library, such as for sequencing. In this manner, the one or more or a plurality of target RNA sequences can be determined in a method of single-cell RNA sequencing that is used to characterize the transcriptome of individual cells within a heterogeneous population.

Aspects of the present disclosure utilize a unique molecular identifier barcode sequence (UMI) of a length between 10 and 30 nucleotides with 20 nucleotides being exemplary. Such a unique molecular identifier barcode sequence length decreases the opportunity for two transcripts having the same UMI. Accordingly, aspects of the present disclosure are directed to associating a different unique molecular identifier barcode sequence for each RNA transcript or its associated cDNA. In this manner, each RNA transcript has its own unique associated unique molecular identifier barcode sequence. In this manner, each RNA transcript within a plurality of RNA transcripts has a different unique molecular identifier barcode sequence from other members of the plurality. Also, such a unique molecular identifier barcode sequence length allows that false UMI sequences (which typically differ only by one or two nucleotides from the true UMI) created by errors in amplification or sequencing of the UMI can be distinguished because the UMI sequences are far apart, i.e., the Hamming distance between UMIs is sufficient to reduce the opportunity for sequencing misreads to be mistaken as distinct UMIs.

Aspects of the present disclosure utilize UMIs with a semi-random pattern as described herein (UMI_{A} and UMI_{B}). The use of semi-random patterns for UMIs allows sequencing or amplification errors to be measured by counting the bases that fall outside the pattern, thereby providing an empirical measurement of sequencing error rate. In particular, insertion or deletion errors in the UMI are readily apparent due to the semi-random pattern. Knowing the error rate is important for understanding the reliability of the UMIs.

According to one aspect. UMI_{A} and UMI_{B} are both 10 to 30 base pair sequences, such as 20 base pair sequences, of semi-random patterns. The pattern for UMI_{A} is [(HBDV)₅] where H=not G, B=not A, D=not C, and V=not T. The pattern for UMI_{B} is [(VDBH)₅]. It is to be understood that other semi-random patterns can be designed. This semirandom pattern provides two advantages. First, amplification or sequencing errors in the UMIs can be detected when bases fall outside the expected pattern, allowing empirical measurement of error rate. Second, since UMI_{B} can be distinguished from UMI_{A}, this allows the exonuclease degradation efficiency to be determined from the ratio of reads with UMI_{A} vs. UMI_{B} incorporated.

Aspects of the present disclosure are directed to methods of measuring the degradation rate of reverse transcription primers (RT-A with UMI_{A} pattern) provided during the reverse transcription method as described herein. Exonuclease digestion improves quantification accuracy by preventing excess reverse transcription primers from binding to DNA. These primers would otherwise attach multiple UMIs to copies of the same mRNA transcript and cause overcounting. According to the method, a reverse transcription primer having a different UMI pattern (RT-B with UMI_{B} pattern) that is distinct from that of the RT-A primer used during RT is added to the mixture post reverse transcription and during the primer degradation step. This allows the measurement of RT primer degradation efficiency as determined by the final ratio of reads of products containing UMI_{A} vs. UMI_{B} patterns.

Aspects of the present disclosure are directed to the use of two cell specific barcodes to label the RNA that originates from each individual cell or sample. The use of two barcodes increases the total number of possible barcode combinations (beyond use of a single barcode) to correlate RNA with a cell or a sample. Two barcode multiplexing allows amplified cDNA from multiple cells to be pooled together for library preparation. Primers incorporate two distinct barcode sequences Cₙ and Gₘ with, for example, 48 and 48 possible sequences respectively (2304 combinations). This minimizes the number of individual library preparations that need to be done and reduces reagent costs. The possible barcode combinations scale quadratically with the number of primers. This is distinguished from barcoding schemes using only one primer, and where a separate primer is needed for every barcode.

Aspects of the present disclosure are directed to methods of making amplicons that are associated with RNA in a sample, where the amplicons are designed to be compatible with standard library preparation kits. The design of the final amplified product is compatible for library preparation with standard kits as described herein which is distinguished from single cell multiplexed amplification methods that require custom library preparation protocols and custom sequencing primers.

The present disclosure provides a method of cDNA synthesis from RNA, such as from a small sample, a single cell or small population of cells. The cDNA can then be amplified using multiple annealing and looping based amplification cycles to produce amplicons include a first cell specific barcode sequence, a second cell specific barcode sequence and a unique molecular identifier barcode sequence. The amplicons can then be sequenced, such as by processing into a sequencing library.

According to one aspect, embodiments provide a three-step procedure that can be performed in a single tube or in a micro-titer plate, for example, in a high throughput format. The first step involves reverse transcribing RNA to cDNA using the primers, reverse transcriptases, nucleases, and other suitable reagents and media described herein or otherwise known to those of skill in the art to produce cDNA having then primer sequence attached thereto. In a second step, the cDNA is amplified using a linear or quasi linear amplification method to produce looped extension products having primer sequences at each end. In a third step, the looped extension products are amplified, for example using PCR primers, reagents and conditions as described herein or as known to those of skill in the art to result in the double stranded amplicons having a first cell specific barcode sequence, a second cell specific barcode sequence and a unique molecular identifier barcode sequence. The cDNA sample in the reaction mixture is subjected to extension or amplification by at least one DNA polymerase, wherein the primers anneal to the DNA to allow the DNA polymerase to synthesize a complementary DNA strand from the 3′ end of the primer to produce a DNA product. The steps for DNA amplification by the DNA polymerase are denaturing the DNA product, if needed; annealing the primers to the DNA to form a DNA-primer hybrid; and incubating the DNA-primer hybrid in the presence of nucleobases to allow the DNA polymerase to extend the primer and synthesize the DNA product.

According to one aspect, the reaction mixture for reverse transcription, extension or amplification forms a single stranded nucleic acid molecule/primer mixture which is a mixture comprising at least one single stranded nucleic acid molecule wherein at least one primer, as described herein, is hybridized to a region in said single stranded nucleic acid molecule. In specific embodiments, multiple primers hybridize to multiple locations of the single stranded nucleic acid molecule. In further specific embodiments, the mixture comprises a plurality of single stranded nucleic acid molecules having multiple degenerate primers hybridized thereto. In additional specific embodiments, the single stranded nucleic acid molecule is cDNA or RNA.

For amplification, the reaction mixture is subjected to a plurality of thermocycles. In a particular thermocycle, the reaction mixture is subjected to a first temperature also known as an annealing temperature for a first period of time to allow for sufficient annealing of the primers to the cDNA sequences. According to this aspect, the primers are annealed to the cDNA sequences at a temperature of below about 30° C. in a first step, such as between about 0′C and about 10° C., The reaction mixture is then subjected to a second temperature also known as an amplification temperature for a second period of time to allow for the amplification of the cDNA sequences. According to this aspect, the cDNA sequences are amplified at a temperature of above about 10′C in a second step, such as between about 10° C. and about 65° C. One of skill will understand that the temperature at which amplification takes place will depend upon the particular polymerase used. For example Φ29 Polymerase is fully active at about 30′C and Bst Polymerase and pyrophage 3173 polymerase (exo-) are fully active about 62′C. The double stranded DNA is then melted at a third temperature, also known as a melting temperature for a third period of time to provide single stranded DNA amplicons which may be used as amplification template. According to this aspect, the double stranded DNA is dehybridized into single stranded DNA at a temperature of above about 90° C. in a third step, such as between about 90° C. and about 100′C.

According to one aspect, looping of an extension product having self-annealing sequences at each end may be carried out at a fourth temperature of between about 55° C. and about 60′C also known as a looping temperature insofar as the self-annealing ends of the extension products anneal together to form a loop. An exemplary temperature is about 58° C.

The final amplification cycle terminates when the reaction mixture is subjected to the melting temperature to produce amplicons for further processing, amplification or sequencing. According to this aspect, the amplicons may be further processed, if in sufficient quantity, for sequencing as described herein. According to an additional aspect, the amplicons may be further amplified for example using standard PCR procedures with buffers, primers and polymerases known to those of skill in the art. According to a still additional aspect, the amplicons may be sequenced, if in sufficient quantity, using high-throughput sequencing methods known to those of skill in the art.

According to certain aspects, the RNA to be amplified is first denatured by heating the reaction mixture to between about 65° C. and about 85° C. and exemplary to about 72° C. for about 10 seconds to about five minutes and exemplary for about three minutes. During this step, the primers may be present in the reaction mixture. Alternatively, the primers can be added to the reaction mixture containing the RNA sample to be amplified before heat denaturation or at any time during the denaturation step or after the heat denaturation step.

The reaction mixture is then cooled and primers are annealed. The temperature of the reaction mixture is lowered to a temperature that allows the primers to anneal to the single-stranded RNA. The annealing temperature of the primers should be between about 0° C. and about 30° C., exemplary between about 0° C. and about 10° C., or about 4° C., for a period of about 10 seconds to about 5 minutes. Next, the reaction temperature is increased to a temperature at which the particular reverse transcriptase is activated and begins to synthesize cDNA. Different reverse transcriptases may become functional at different temperatures, such that the cycle can ramp up or increase in temperature such that reverse transcriptases can be activated in series to begin to synthesize cDNA. The total incubation period may be between about 2 minutes to about 15 minutes, more preferably about 10 minutes. It is to be understood that temperatures, incubation periods and ramp times of the reverse transcription step may vary from the values disclosed herein without significantly altering the efficiency of cDNA production. Those of skill in the art will understand based on the present disclosure that parameters can be varied. Minor variations in reaction conditions and parameters are included within the scope of the present disclosure.

The cDNA to be amplified in the first set of reactions is heated to between about 70° C. and about 90° C., and exemplary to about 80° C. for about 10 seconds to about five minutes and exemplary for about two minutes to degrade the RNA. During this step, primers may be present in the reaction mixture. Alternatively, the primers can be added to the reaction mixture containing the cDNA sample after the RNA is degraded.

For amplification of the looped extension products, the temperature of the reaction mixture is raised to denature the looped extension products into single stranded form. The temperature is lowered to a temperature that allows the primers to anneal to the cDNA. The annealing temperature of the primers is between about 0′C and about 30′C, exemplary between about 0′C and about 10° C. for a period of about 10 seconds to about 5 minutes. Next, the reaction temperature is increased to a temperature at which the particular DNA polymerase becomes activated and begins to synthesize DNA. Different DNA polymerases may become functional at different temperatures, such that the cycle can ramp up or increase in temperature such that different DNA polymerases can be activated in series to begin to synthesize DNA. The total incubation period may be between about 2 minutes to about 7 minutes, more preferably about 5 minutes.

It is to be understood that temperatures, incubation periods and ramp times of the DNA amplification steps may vary from the values disclosed herein without significantly altering the efficiency of DNA amplification. Those of skill in the art will understand based on the present disclosure that parameters can be varied. Minor variations in reaction conditions and parameters are included within the scope of the present disclosure.

The resulting amplicons can then be processed for sequencing as described herein or as known to those of skill in the art.

### RNA, Cell Type and Sample

The term “RNA” as used herein may be understood by one of skill in the art to refer to a polymeric molecule essential in various biological roles in coding, decoding, regulation, and expression of genes. RNA, like DNA, is a nucleic acid. RNA is assembled as a chain of nucleotides and is often found as a single-strand folded onto itself into a secondary structure. RNA generally includes the nucleotides G, U, A, and C to denote the nitrogenous bases guanine, uracil, adenine, and cytosine. Types of RNA include messenger RNA, transfer RNA, ribosomal RNA, long noncoding RNA, small interfering RNA, and other RNA types known to those of skill in the art.

According to one aspect, the RNA is messenger RNA or other RNA from natural or artificial sources to be tested. In another preferred embodiment, the RNA sample is mammalian RNA, plant RNA, yeast RNA, viral RNA, or prokaryotic RNA. In yet another preferred embodiment, the RNA sample is obtained from a human, bovine, porcine, ovine, equine, rodent, avian, fish, shrimp, plant, yeast, virus, or bacteria. Preferably the RNA sample is messenger RNA from a single cell.

According to one aspect, the RNA is from a single cell. According to one aspect, the RNA is from a single cell within a heterogeneous population of cells. According to one aspect, the RNA is from a single prenatal cell. According to one aspect, the RNA is from a single cancer cell. According to one aspect, the RNA is from a single circulating tumor cell.

The term “isolated RNA” (e.g., “isolated mRNA”) refers to RNA molecules which are substantially free of other cellular material, or culture medium when produced by recombinant techniques, or substantially free of chemical precursors or other chemicals when chemically synthesized.

According to one aspect, the sample may be in vitro. The term “in vitro” has its art recognized meaning. e.g., involving purified reagents or extracts. e.g., cell extracts.

As used herein, the term “biological sample” is intended to include, but is not limited to, tissues, cells, biological fluids and isolates thereof, isolated from a subject, as well as tissues, cells and fluids present within a subject.

RNA processed by methods described herein may be obtained from any useful source, such as, for example, a human sample. The sample may be any sample from a human, such as blood, serum, plasma, cerebrospinal fluid, cheek scrapings, nipple aspirate, biopsy, semen (which may be referred to as ejaculate), urine, feces, hair follicle, saliva, sweat, immunoprecipitated or physically isolated chromatin, and so forth. In specific embodiments, the sample comprises a single cell. In specific embodiments, the sample includes only a single cell.

In particular embodiments, the amplified nucleic acid molecule from the sample provides diagnostic or prognostic information. For example, the prepared nucleic acid molecule from the sample may provide genomic copy number and/or sequence information, allelic variation information, cancer diagnosis, prenatal diagnosis, paternity information, disease diagnosis, detection, monitoring, and/or treatment information, sequence information, and so forth.

As used herein, a “single cell” refers to one cell. Single cells useful in the methods described herein can be obtained from a tissue of interest, or from a biopsy, blood sample, or cell culture. Additionally, cells from specific organs, tissues, tumors, neoplasms, or the like can be obtained and used in the methods described herein. Furthermore, in general, cells from any population can be used in the methods, such as a population of prokaryotic or eukaryotic single celled organisms including bacteria or yeast. A single cell suspension can be obtained using standard methods known in the art including, for example, enzymatically using trypsin or papain to digest proteins connecting cells in tissue samples or releasing adherent cells in culture, or mechanically separating cells in a sample. Single cells can be placed in any suitable reaction vessel in which single cells can be treated individually. For example, a 96-well plate, such that each single cell is placed in a single well.

Cells within the scope of the present disclosure include any type of cell where understanding the RNA content is considered by those of skill in the art to be useful. A cell according to the present disclosure includes a cancer cell of any type, hepatocyte, oocyte, embryo, stem cell, iPS cell. ES cell, neuron, erythrocyte, melanocyte, astrocyte, germ cell, oligodendrocyte, kidney cell and the like. According to one aspect, the methods of the present invention are practiced with the cellular RNA from a single cell. A plurality of cells includes from about 2 to about 1,000,000 cells, about 2 to about 10 cells, about 2 to about 100 cells, about 2 to about 1,000 cells, about 2 to about 10,000 cells, about 2 to about 100,000 cells, about 2 to about 10 cells or about 2 to about 5 cells.

Methods for manipulating single cells are known in the art and include fluorescence activated cell sorting (FACS), flow cytometry (Herzenberg., PNAS USA 76:1453-55 1979), micromanipulation and the use of semi-automated cell pickers (e.g. the Quixell™ cell transfer system from Stoelting Co.). Individual cells can, for example, be individually selected based on features detectable by microscopic observation, such as location, morphology, or reporter gene expression. Additionally, a combination of gradient centrifugation and flow cytometry can also be used to increase isolation or sorting efficiency.

Once a desired cell has been identified, the cell is lysed to release cellular contents including RNA, using methods known to those of skill in the art. The cellular contents are contained within a vessel or a collection volume. In some aspects of the invention, cellular contents, such as RNA, can be released from the cells by lysing the cells. Lysis can be achieved by, for example, heating the cells, or by the use of detergents or other chemical methods, or by a combination of these. However, any suitable lysis method known in the art can be used. For example, heating the cells at 72° C. for 2 minutes in the presence of Tween-20 is sufficient to lyse the cells. Alternatively, cells can be heated to 65° C. for 10 minutes in water (Esumi et al., Neurosci Res 60(4):439-51 (2008)); or 70′C for 90 seconds in PCR buffer II (Applied Biosystems) supplemented with 0.5% NP-40 (Kurimoto et al., Nucleic Acids Res 34(5):e42 (2006)); or lysis can be achieved with a protease such as Proteinase K or by the use of chaotropic salts such as guanidine isothiocyanate (U.S. Publication No. 2007/0281313). Amplification of RNA according to methods described herein can be performed directly on cell lysates, such that a reaction mix can be added to the cell lysates. Alternatively, the cell lysate can be separated into two or more volumes such as into two or more containers, tubes or regions using methods known to those of skill in the art with a portion of the cell lysate contained in each volume container, tube or region. RNA contained in each container, tube or region may then be amplified by methods described herein or methods known to those of skill in the art.

cDNA Synthesis from RNA

Methods described herein utilize “reverse-transcriptase PCR” (“RT-PCR”) which is a type of PCR where the starting material is mRNA. The starting mRNA is enzymatically converted to complementary DNA or “cDNA” using a reverse transcriptase enzyme. The cDNA is then used as a template for a PCR reaction.

According to one aspect, cDNA is generated from RNA wherein the resulting cDNA includes a first cell specific barcode sequence and a first unique molecular identifier barcode sequence. According to one aspect. cDNA is synthesized from an RNA template, such as a mRNA template obtained, i.e. lysed, from a single cell. In a reaction vessel, the RNA template is denatured from its secondary structure into a single stranded form. Reverse transcription primer sequences are added having 3′ poly(T) sequences complementary to the 5′ poly(A) sequences of RNA template strands. The reverse transcription primer sequence further includes a 5′ self-annealing sequence, a barcode primer annealing site, a first cell specific barcode sequence having between 4 and 12 nucleotides and a first unique molecular identifier barcode sequence having between 10 to 30 nucleotides. For a given mRNA, the 3′ poly(T) sequence of the reverse transcription primer sequence, which may include between 10 to 30 T nucleotides, hybridizes to the 5′ poly(A) sequence of the RNA template strand.

In the presence of a reverse transcriptase and under suitable conditions and reagents, the RNA template strands are reverse transcribed to produce cDNA template strands including the reverse transcription primer sequence 5′ of the cDNA template strand. The cDNA template strand is hybridized to the RNA strand. Excess reverse transcription primer sequences are digested, such as with a digestion enzyme. The RNA strand is degraded to produce the cDNA template strand as a single strand. The reverse transcriptase is inactivated. The digestion enzyme is inactivated. The resulting cDNA is then amplified.

A reverse transcriptase (RT) is an enzyme used to generate complementary DNA (cDNA) from an RNA template, a process termed reverse transcription. According to one aspect, exemplary and useful reverse transcriptases are commercially available and/or known to those of skill in the art. A reverse transcriptase applies the polymerase chain reaction technique to RNA in a technique called reverse transcription polymerase chain reaction (RT-PCR). Reverse transcriptase is used in the present disclosure to create cDNA libraries from mRNA. An exemplary reverse transcriptase is commercially available as SuperScript II, III or IV, M-MLV Reverse Transcriptase, Maxima Reverse Transcriptase, Protoscript Reverse Reverse Transcriptase. Thermoscript Reverse Transcriptase, or numerous other compatible, known or commercially available reverse transcriptases.

Enzymes used to digest primers are known to those of skill in the art and are commercially available. Exemplary digestion enzymes include Exonuclease 1, Exonuclease I with shrimp alkaline phosphatase, Exonuclerase T and other suitable nucleases and the like.

According to the cDNA synthesis method described above, the reaction media in the reaction vessel is subjected to several temperatures to accomplish various aspects of the method. For example, the RNA strand is degraded at a temperature of between 75° C. and 85° C. The reverse transcriptase and the enzyme are inactivated at a temperature of between 75° C. and 85° C.

cDNA Amplification Using Multiple Annealing and Looping Based Amplification Cycles

The resulting single stranded cDNA molecules are then amplified using multiple annealing and looping based amplification cycles. According to one aspect, complementary strands to the cDNA template strands including the reverse transcription primer sequence are generated using a DNA polymerase under suitable conditions and reagents including an extension primer including the self-annealing sequence at the 5′ end of the primer. The resulting complementary strands include the self-annealing sequence at the 5′ end and its complement at the 3′ end. The cDNA template strands are denatured from the complementary strands and the complementary are looped by annealing of the self-annealing sequence at the 3′ end and its complement at the 5′ end. Once looped, the looped complementary strands are inhibited from being amplified. The steps of generating the complementary strands to the cDNA template and denaturing the cDNA strands from the complementary strands followed by looping of the complementary strands are repeated a plurality of times, such as between 7 and 12 times to generate a plurality of looped complementary strands from each cDNA template strand.

The plurality of looped complementary strands are denatured and then amplified using an amplification primer including the self-annealing sequence to produce double stranded amplicons including the reverse transcription primer sequence. The double stranded amplicons are denatured and repeatedly amplified a plurality of times using (1) an outer barcode primer having a 3′ sequence complementary to the barcode primer annealing site, wherein the outer barcode primer further includes a 5′ self-annealing sequence, a sequencing priming sequence and a second cell specific barcode sequence having between 4 and 12 nucleotides, and (2) a primer including a 5′ self-annealing sequence. The resulting double stranded amplicons include a first cell specific barcode sequence, a second cell specific barcode sequence and a first unique molecular identifier barcode sequence. The resulting double stranded amplicons are processed for sequencing.

According to one aspect, the first unique molecular identifier barcode sequence may have a semi-random sequence pattern.

Exemplary self-annealing sequences are known to those of skill in the art and include is GAT5 and GAT1 and the like.

Exemplary barcode primer annealing site sequences are known to those of skill in the art and include RT3, Read2SP, Read1SP and the like.

According to one aspect, a reaction mixture of one or more or a plurality of cDNA sequences reverse transcribed from one or more or a plurality of RNA sequences, primers and at least one polymerase is provided. According to one aspect, the polymerase has strand displacement activity or has 5′ to 3′ exonuclease activity is provided. Strand-displacing polymerases are polymerases that will dislocate downstream fragments as it extends. Strand displacing polymerases include Φ29 Polymerase, Bst Polymerase, Pyrophage 3173. Vent Polymerase, Deep Vent polymerase, TOPO Taq DNA polymerase, Taq polymerase, T7 polymerase, Vent (exo-) polymerase, Deep Vent (exo-) polymerase, 9°Nm Polymerase, Klenow fragment of DNA Polymerase I, MMLV Reverse Transcriptase. AMV reverse transcriptase, HIV reverse transcriptase, a mutant form of T7 phage DNA polymerase that lacks 3′-5′ exonuclease activity, or a mixture thereof. One or more polymerases that possess a 5′ flap endonuclease or 5′-3′ exonuclease activity such as Taq polymerase, Bst DNA polymerase (full length), E. coli DNA polymerase, LongAmp Taq polymerase, OneTaq DNA polymerase or a mixture thereof may be used to remove residual bias due to uneven priming. Other polymerases that do not have strand displacement activity are useful, such as Q5, Phusion and Kapa HiFi.

Sequencing priming sequences, adapter sequences, sequencing indexes, flowcell annealing adapters useful for preparing a sequencing library are known to those of skill in the art and are commercially available and include Read1SP, Read2SP, Index1, Index2. P5, and P7.

Exemplary sequences are provided in Table 1 below. All sequences are listed from 5′ to 3′. H=not G, B=not A, D=not C, V=not T. The sequences of Read1SP, Read2SP, Index1, Index2, P5, and P7 are known to those of skill in the art and are available from Illumina and Ilumina published information.

| Sequence | |
|---|---|
| Name | Nucleotide Sequence |
| GAT5 | GTAGGTGTGAGTGATGGTTGAGGTAGT |
| B1 | GAGGAG |
| GAT1 | GTGAGTGATGGTTGAGGTAGTGTGGAG |
| RT3 | AGTCGCTTGGGTGTAGTGC |
| UMI_{A} | HBDVHBDVHBDVHBDVHBDV |
| UMI_{B} | VDBHVDBHVDBHVDBHVDBH |
| Cₙ | GTTGTT, GTTAAA, GTTTGG, AGGGTT, AGGAAA, |
| | AGGTGG, TAATGG, GGAGAG, GGAAGT, GGATTA, |
| | AATGAG, AATAGT, AATTTA, TTGGAG, TTGAGT, |
| | TTGTTA, ATAATG, ATATAT, ATAGGA, TGTATG, |
| | TGTTAT, TGTGGA, GAGATG, GAGTAT, GAGGGA, |
| | GTTGAG, GTTAGT, GTTTTA, AGGGAG, AGGAGT, |
| | AGGTTA, TAAGAG, TAAAGT, TAATTA, GTTATG, |
| | GTTTAT, GTTGGA, AGGATG, AGGTAT, AGGGGA, |
| | TAAATG, TAATAT, TAAGGA, GGAGTT, AATGTT, |
| | TTGGTT, GGAAAA, AATAAA |
| Gₘ | GATATG, ATACG, CCGTCTG, TGCG, GAACTCG, |
| | ATGTAG, CCCG, TGTAG, GAGTAAG, ATCG, |
| | CCTAG, TGACCG, GACG, ATTAG, CCACTG, |
| | TGGTCTG, GTTTACG, ACAG, CGGAG, TACCTG, |
| | GTAG, ACGACG, CGCCG, TATTAAG, GTGATCG, |
| | ACCCG, CGTTCG, TAAG, GTCCG, ACTTATG, |
| | CGAG, TAGATG, GCTCAG, AGATG, CAGG, |
| | TTCACAG, GCAATCG, AGGCCG, CACTG, TTTG, |
| | GCGG, AGCAG, CATCTG, TTATATG, GCCTG, |
| | AGTG, CAAACG, TTGCAAG |

According to the multiple annealing and looping based amplification cycles method described above, the reaction media in the reaction vessel is subjected to several temperatures to accomplish various aspects of the method. For example, the extension primer anneals to the cDNA template strand at a temperature of between 0° C. and 10° C. The complementary strand is generated at a temperature of between 10° C. and 65° C. Looping the complementary strand occurs at a temperature of between 55° C. and 600° C.

According to one aspect, the step of amplifying the denatured complementary strands is carried out using polymerase chain reaction, such as using between 15 and 20 cycles of polymerase chain reaction.

According to one aspect, the step of amplifying the denatured amplicons is carried out using polymerase chain reaction, such as using between 3 and 7 cycles of polymerase chain reaction.

According to one aspect, the sequencing priming sequence is Read2SP or Read1SP.

### Measuring Reverse Transcription Primer Degradation Efficiency

According to one aspect, a method is provided for measuring or otherwise determining the efficiency of reverse transcription primer degradation efficiency. The method includes adding reverse transcription primers with second unique molecular identifier barcode sequences having between 10 to 30 nucleotides in the presence of the digestion enzyme. The second unique molecular identifier barcode sequences include a semi-random sequence pattern which is different from the first unique molecular identifier barcode sequence. In this manner, the RT primer degradation efficiency can be measured in terms of the final ratio of products including the first unique molecular identifier barcode sequences and the second unique molecular identifier barcode sequences.

### Amplification

In certain aspects, amplification is achieved using PCR. PCR is a reaction in which replicate copies are made of a target polynucleotide using a pair of primers or a set of primers consisting of an upstream and a downstream primer, and a catalyst of polymerization, such as a DNA polymerase, and typically a thermally-stable polymerase enzyme. Methods for PCR are well known in the art, and taught, for example in MacPherson et al. (1991) PCR 1: A Practical Approach (IRL Press at Oxford University Press). The term “polymerase chain reaction” (“PCR”) of Mullis (U.S. Pat. Nos. 4,683,195, 4,683,202, and 4,965,188) refers to a method for increasing the concentration of a segment of a target sequence without cloning or purification. This process for amplifying the target sequence includes providing oligonucleotide primers with the desired target sequence and amplification reagents, followed by a precise sequence of thermal cycling in the presence of a polymerase (e.g., DNA polymerase). The primers are complementary to their respective strands (“primer binding sequences”) of the double stranded target sequence. In general, to effect amplification, the double stranded target sequence is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle;” there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”) and the target sequence is said to be “PCR amplified.”

The terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

Any oligonucleotide or polynucleotide sequence can be amplified with the appropriate set of primer molecules. Methods and kits for performing PCR are well known in the art. All processes of producing replicate copies of a polynucleotide, such as PCR or gene cloning, are collectively referred to herein as replication.

The expression “amplification” or “amplifying” refers to a process by which extra or multiple copies of a particular polynucleotide are formed. Amplification includes methods such as PCR, ligation amplification (or ligase chain reaction, LCR) and other amplification methods. These methods are known and widely practiced in the art. See, e.g., U.S. Pat. Nos. 4,683,195 and 4,683,202 and Innis et al., “PCR protocols: a guide to method and applications” Academic Press, Incorporated (1990) (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization. i.e. each primer is specifically designed to be complementary to each strand of the genomic locus to be amplified.

Reagents and hardware for conducting amplification reactions are commercially available. Primers useful to amplify sequences from a particular gene region are preferably complementary to, and hybridize specifically to sequences in the target region or in its flanking regions and can be prepared using methods known to those of skill in the art. Nucleic acid sequences generated by amplification can be sequenced directly.

When hybridization occurs in an antiparallel configuration between two single-stranded polynucleotides, the reaction is called “annealing” and those polynucleotides are described as “complementary”. A double-stranded polynucleotide can be complementary or homologous to another polynucleotide, if hybridization can occur between one of the strands of the first polynucleotide and the second. Complementarity or homology (the degree that one polynucleotide is complementary with another) is quantifiable in terms of the proportion of bases in opposing strands that are expected to form hydrogen bonding with each other, according to generally accepted base-pairing rules.

The term “amplification reagents” may refer to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.). Amplification methods include PCR methods known to those of skill in the art and also include rolling circle amplification (Blanco et al., J. Biol. Chem., 264, 8935-8940, 1989), hyperbranched rolling circle amplification (Lizard et al., Nat. Genetics, 19, 225-232, 1998), and loop-mediated isothermal amplification (Notomi et al., Nuc. Acids Res., 28, e63, 2000) each of which are hereby incorporated by reference in their entireties.

Other amplification methods, as described in British Patent Application No. GB 2,202,328, and in PCT Patent Application No. PCT/US89/01025, each incorporated herein by reference, may be used in accordance with the present disclosure. Emulsion PCR may be used in accordance with the present disclosure. Other suitable amplification methods include “race and “one-sided PCR.”. (Frohman, In: PCR Protocols: A Guide To Methods And Applications, Academic Press, N.Y., 1990, each herein incorporated by reference). Methods based on ligation of two (or more) oligonucleotides in the presence of nucleic acid having the sequence of the resulting “di-oligonucleotide,” thereby amplifying the di-oligonucleotide, also may be used to amplify DNA in accordance with the present disclosure (Wu et al., Genomics 4:560-569, 1989, incorporated herein by reference).

RNA to be amplified may be obtained from a single cell or a small population of cells. Methods described herein allow RNA to be amplified from any species or organism in a reaction mixture, such as a single reaction mixture carried out in a single reaction vessel. In one aspect, methods described herein include sequence independent amplification of RNA from any source including but not limited to human, animal, plant, yeast, viral, eukaryotic and prokaryotic RNA.

### Primers

As used herein, the term “primer” generally includes an oligonucleotide, either natural or synthetic, that is capable, upon forming a duplex with a polynucleotide template, of acting as a point of initiation of nucleic acid synthesis, such as a sequencing primer, and being extended from its 3′ end along the template so that an extended duplex is formed. Primers include extension primers, amplification primers or reverse transcription primers.

The sequence of nucleotides added during the extension process is determined by the sequence of the template polynucleotide. Usually primers are extended by a DNA polymerase or reverse transcriptase. Primers usually have a length in the range of between 3 to 36 nucleotides, also 5 to 24 nucleotides, also from 14 to 36 nucleotides. Primers within the scope of the invention include orthogonal primers, amplification primers, constructions primers and the like. Pairs of primers can flank a sequence of interest or a set of sequences of interest. Primers and probes can be degenerate or quasi-degenerate in sequence. Primers within the scope of the present invention bind adjacent to a target sequence. A “primer” may be considered a short polynucleotide, generally with a free 3′-OH group that binds to a target or template potentially present in a sample of interest by hybridizing with the target, and thereafter promoting polymerization of a polynucleotide complementary to the target. Primers of the instant invention are comprised of nucleotides ranging from 17 to 30 nucleotides. In one aspect, the primer is at least 17 nucleotides, or alternatively, at least 18 nucleotides, or alternatively, at least 19 nucleotides, or alternatively, at least 20 nucleotides, or alternatively, at least 21 nucleotides, or alternatively, at least 22 nucleotides, or alternatively, at least 23 nucleotides, or alternatively, at least 24 nucleotides, or alternatively, at least 25 nucleotides, or alternatively, at least 26 nucleotides, or alternatively, at least 27 nucleotides, or alternatively, at least 28 nucleotides, or alternatively, at least 29 nucleotides, or alternatively, at least 30 nucleotides, or alternatively at least 50 nucleotides, or alternatively at least 75 nucleotides or alternatively at least 100 nucleotides.

### Sequencing

The amplicons are sequenced using, for example, high-throughput sequencing methods known to those of skill in the art. Determination of the sequence of a nucleic acid sequence of interest can be performed using a variety of sequencing methods known in the art including, but not limited to, sequencing by hybridization (SBH), sequencing by ligation (SBL) (Shendure et al. (2005) Science 309:1728), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads (U.S. Pat. No. 7,425,431), wobble sequencing (PCT/US05/27695), multiplex sequencing (U.S. Ser. No. 12/027,039, filed Feb. 6, 2008; Porreca et al (2007) Nat. Methods 4:931), polymerized colony (POLONY) sequencing (U.S. Pat. Nos. 6,432,360, 6,485,944 and 6,511,803, and PCT/US05/06425); nanogrid rolling circle sequencing (ROLONY) (U.S. Ser. No. 12/120,541, filed May 14, 2008), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout) and the like. High-throughput sequencing methods, e.g., using platforms such as Roche 454. Illumina Solexa, AB-SOLiD, Helicos, Polonator platforms and the like, can also be utilized. A variety of light-based sequencing technologies are known in the art (Landegren et al. (1998) Genome Res. 8:769-76; Kwok (2000) Pharmacogenomics 1:95-100; and Shi (2001) Clin. Chem. 47:164-172).

The amplified DNA can be sequenced by any suitable method. In particular, the amplified DNA can be sequenced using a high-throughput screening method, such as Applied Biosystems' SOLiD sequencing technology, or Illumina's Genome Analyzer. In one aspect of the invention, the amplified DNA can be shotgun sequenced. The number of reads can be at least 10,000, at least 1 million, at least 10 million, at least 100 million, or at least 1000 million. In another aspect, the number of reads can be from 10.000 to 100,000, or alternatively from 100.000 to 1 million, or alternatively from 1 million to 10 million, or alternatively from 10 million to 100 million, or alternatively from 100 million to 1000 million. A “read” is a length of continuous nucleic acid sequence obtained by a sequencing reaction.

“Shotgun sequencing” refers to a method used to sequence very large amount of DNA (such as the entire genome). In this method, the DNA to be sequenced is first shredded into smaller fragments which can be sequenced individually. The sequences of these fragments are then reassembled into their original order based on their overlapping sequences, thus yielding a complete sequence. “Shredding” of the DNA can be done using a number of difference techniques including restriction enzyme digestion or mechanical shearing. Overlapping sequences are typically aligned by a computer suitably programmed. Methods and programs for shotgun sequencing a cDNA library are well known in the art.

The amplification and sequencing methods are useful in the field of predictive medicine in which diagnostic assays, prognostic assays, pharmacogenomics, and monitoring clinical trials are used for prognostic (predictive) purposes to thereby treat an individual prophylactically. Accordingly, one aspect of the present invention relates to diagnostic assays for determining the RNA in order to determine whether an individual is at risk of developing a disorder and/or disease. Such assays can be used for prognostic or predictive purposes to thereby prophylactically treat an individual prior to the onset of the disorder and/or disease. Accordingly, in certain exemplary embodiments, methods of diagnosing and/or prognosing one or more diseases and/or disorders using one or more of expression profiling methods described herein are provided.

### Complementarity and Hybridization

As used herein, the terms “complementary” and “complementarity” are used in reference to nucleotide sequences related by the base-pairing rules. For example, the sequence 5′-AGT-3′ is complementary to the sequence 5′-ACT-3′. Complementarity can be partial or total. Partial complementarity occurs when one or more nucleic acid bases is not matched according to the base pairing rules. Total or complete complementarity between nucleic acids occurs when each and every nucleic acid base is matched with another base under the base pairing rules. The degree of complementarity between nucleic acid strands has significant effects on the efficiency and strength of hybridization between nucleic acid strands.

The term “hybridization” refers to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

The term “Tₘ” refers to the melting temperature of a nucleic acid. The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41 (% G+C), when a nucleic acid is in aqueous solution at 1 M NaCl (See. e.g., Anderson and Young, Quantitative Filter Hybridization, in Nucleic Acid Hybridization (1985)). Other references include more sophisticated computations that take structural as well as sequence characteristics into account for the calculation of Tₘ.

The term “stringency” refers to the conditions of temperature, ionic strength, and the presence of other compounds such as organic solvents, under which nucleic acid hybridizations are conducted.

“Low stringency conditions,” when used in reference to nucleic acid hybridization, comprise conditions equivalent to binding or hybridization at 42° C. in a solution consisting of 5×SSPE (43.8 g/l NaCl, 6.9 g/l μl NaH₂PO₄(H₂O) and 1.85 g/l EDTA. pH adjusted to 7.4 with NaOH), 0.1% SDS, 5×Denhardt's reagent (50×Denhardt's contains per 500 ml: 5 g Ficoll (Type 400, Pharmacia), 5 g BSA (Fraction V; Sigma)) and 100 mg/ml denatured salmon sperm DNA followed by washing in a solution comprising 5×SSPE, 0.1% SDS at 42° C. when a probe of about 500 nucleotides in length is employed.

“Medium stringency conditions,” when used in reference to nucleic acid hybridization, comprise conditions equivalent to binding or hybridization at 42° C. in a solution consisting of 5×SSPE (43.8 g/l NaCl, 6.9 g/l NaH₂PO₄(H₂O) and 1.85 g/l EDTA. pH adjusted to 7.4 with NaOH), 0.5% SDS, 5×Denhardt's reagent and 100 mg/ml denatured salmon sperm DNA followed by washing in a solution comprising 1.0×SSPE, 1.0% SDS at 42° C. when a probe of about 500 nucleotides in length is employed.

“High stringency conditions,” when used in reference to nucleic acid hybridization, comprise conditions equivalent to binding or hybridization at 42° C. in a solution consisting of 5×SSPE (43.8 g/l μl NaCl, 6.9 g/l NaH₂PO₄(H₂O) and 1.85 g/l EDTA, pH adjusted to 7.4 with NaOH), 0.5% SDS, 5×Denhardt's reagent and 100 mg/ml denatured salmon sperm DNA followed by washing in a solution comprising 0.1×SSPE, 1.0% SDS at 42° C. when a probe of about 500 nucleotides in length is employed.

### Software and Electronic Apparatuses and Media

In certain exemplary embodiments, electronic apparatus readable media comprising one or more RNA or cDNA sequences described herein is provided. As used herein, “electronic apparatus readable media” refers to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; general hard disks and hybrids of these categories such as magnetic/optical storage media. The medium is adapted or configured for having recorded thereon one or more expression profiles described herein.

As used herein, the term “electronic apparatus” is intended to include any suitable computing or processing apparatus or other device configured or adapted for storing data or information. Examples of electronic apparatuses suitable for use with the present invention include stand-alone computing apparatus; networks, including a local area network (LAN), a wide area network (WAN) Internet, Intranet, and Extranet; electronic appliances such as a personal digital assistants (PDAs), cellular phone, pager and the like; and local and distributed processing systems.

As used herein. “recorded” refers to a process for storing or encoding information on the electronic apparatus readable medium. Those skilled in the art can readily adopt any of the presently known methods for recording information on known media to generate manufactures comprising one or more expression profiles described herein.

A variety of software programs and formats can be used to store the RNA or cDNA information of the present invention on the electronic apparatus readable medium. For example, the nucleic acid sequence can be represented in a word processing text file, formatted in commercially-available software such as WordPerfect and MicroSoft Word, or represented in the form of an ASCII file, stored in a database application, such as DB2, Sybase, Oracle, or the like, as well as in other forms. Any number of data processor structuring formats (e.g., text file or database) may be employed in order to obtain or create a medium having recorded thereon one or more expression profiles described herein.

It is to be understood that the embodiments of the present invention which have been described are merely illustrative of some of the applications of the principles of the present invention. Numerous modifications may be made by those skilled in the art based upon the teachings presented herein without departing from the true spirit and scope of the invention. The contents of all references, patents and published patent applications cited throughout this application are hereby incorporated by reference in their entirety for all purposes.

The following examples are set forth as being representative of the present invention. These examples are not to be construed as limiting the scope of the invention as these and other equivalent embodiments will be apparent in view of the present disclosure, figures and accompanying claims.

### Example I

### cDNA Synthesis from mRNA Template

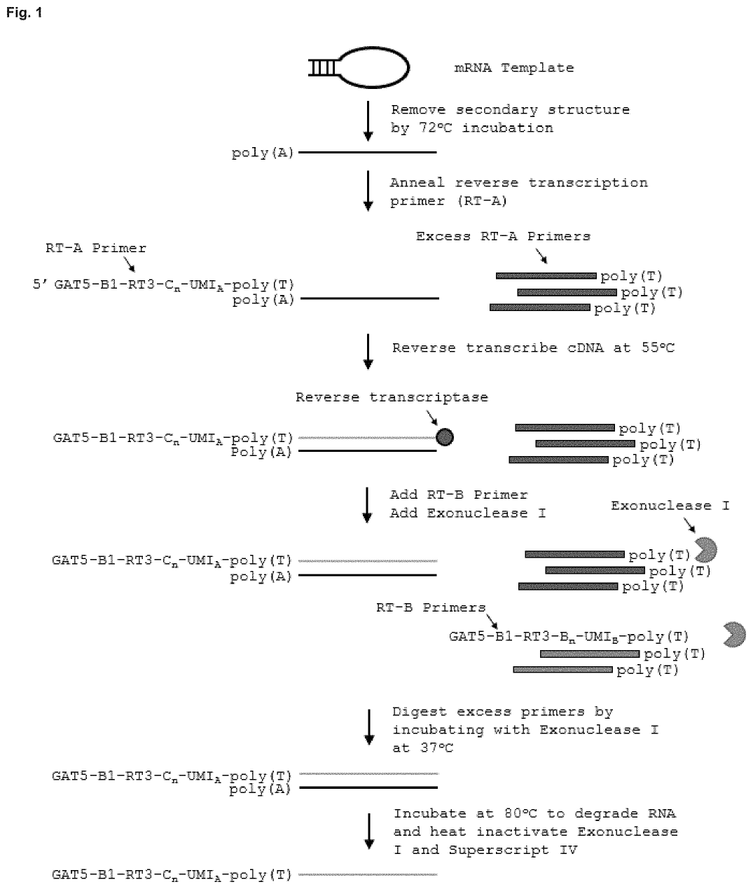

FIG. 1 illustrates one exemplary method for synthesizing cDNA from a mRNA template. Lysed RNA suspended in 4 μl of cell lysis buffer (1× SuperScript IV Buffer (Thermo Fisher Scientific), 0.5% IGEPAL CA-630 (Sigma-Aldrich), 500 mM dNTP, 6 mM MgSO₄, 1M Betaine, 1U SUPERase In RNase Inhibitor (Thermo Fisher Scientific), 2.5 μM ‘RT-A’ reverse transcription primer (IDT)) is heated to 72° C. for 3 minutes to denature RNA secondary structure. After heating, the mixture is cooled to 4° C. to anneal the reverse transcriptase primer (“RT-A) to the poly(A) tract of the mRNA transcript. The RT-A primer contains (starting from the 5′ end) the GAT5 sequence, which is used to create self-annealing loops during cDNA amplification, the B1 spacer sequence, the RT3 sequence, which is used as an annealing site for the outer barcode primer during the final PCR step, the Cₙ sequence, which is one of ‘n’ different 6 nucleotide cell specific barcodes separated by ≥3 Hamming distance, the UMI_{A} sequence, which is a reduced complexity, i.e. semi-random, 20-mer with ˜3.5 billion (3²⁰) possible combinations to uniquely barcode each transcript, and a 12-nucleotide poly(T) tract (see Table 1), 20 μl of reverse transcriptase mix (1× SuperScript IV Buffer, 0.1M DTT, 1U SUPERase In RNase Inhibitor, 60U SuperScript IV (Thermo Fisher Scientific)) is added and the mixture incubated at 55° C. for 10 minutes to catalyze cDNA synthesis. To prevent excess RT-A primers from annealing during later cDNA amplification, 2 μl primer digestion mix (1× Exonuclease I Buffer (NEB), 12U Exonuclease 1 (NEB), 2.5 uM ‘RT-B’ reverse transcription primer (IDT)) is added and incubated at 37° C. for 30 minutes to digest reverse transcription primers. According to one aspect, a second reverse transcription primer (“RT-B) is added and it is identical to RT-A except it contains the UMI_{B} pattern instead of the UMI_{A} pattern (see Table 1), which allows exonuclease digestion efficiency to be measured since incomplete digestion will result in cDNA amplification products with a mixture of UMI_{A} and UMIs barcodes. Following digestion, the mixture is heated to 80° C. for 20 minutes to degrade the RNA and heat inactivate Exonuclease I and SuperScript IV.

### Example II

### cDNA Amplification

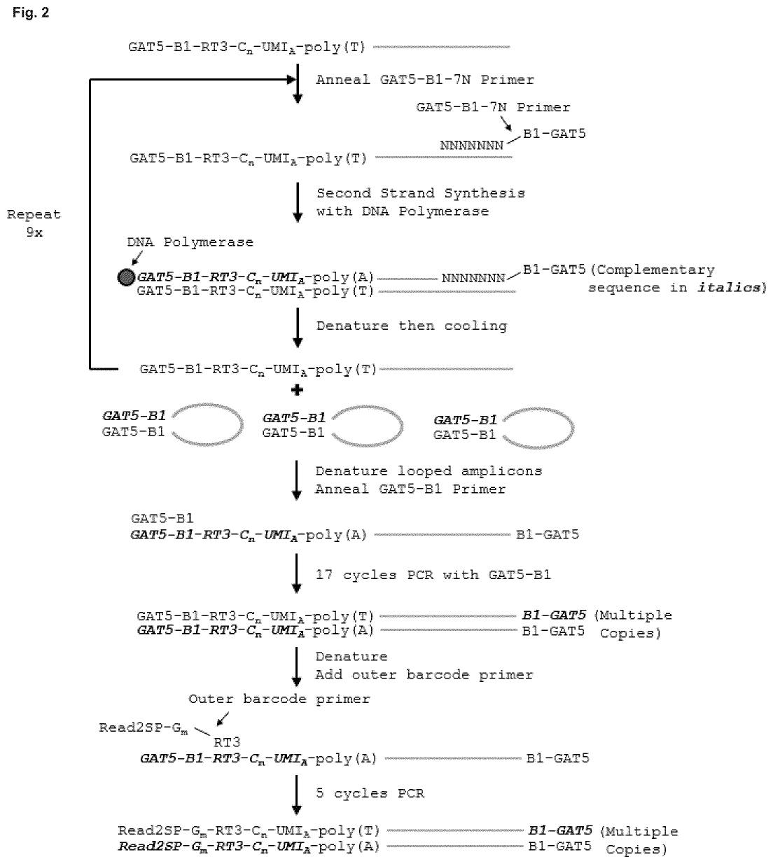

FIG. 2 illustrates amplification of the cDNA of Example I using multiple annealing and looping based amplification cycles (MALBAC) to form looped extension products followed by PCR amplification of the looped extension products. The MALBAC process is described at Zong, C., Lu, S., Chapman, A. R. and Xie, X. S. (2012) Genome-wide detection of single-nucleotide and copy-number variations of a single human cell. Science, 338, 1622-1626; and Chapman. A. R., He. Z., Lu. S., Yong, J., Tan. L., Tang. F. and Xie. X. S. (2015) Single cell transcriptome amplification with MALBAC. PLoS One, 10, e0120889 each of which are hereby incorporated by reference in its entirety.

For MALBAC, 22 μl of cDNA amplification mix (1× ThermoPol buffer (NEB), 200 μM dNTP, 1.25 mM MgSO₄, 50 μM ‘GAT5-B1-7N’ primer (IDT), 50 μM ‘GAT5-B1’ primer (IDT), 2U Deep Vent (exo-) DNA Polymerase (NEB)) is added to the cDNA synthesis mix. The mixture is heated to 95° C. for 5 minutes, then quasilinear cDNA amplification is conducted by repeating the following incubation program 10 times: 4° C. for 50 s, 10° C. for 50 s, 20° C. for 50 s, 30′C for 50 s, 40° C. for 45 s, 50′C for 45 s, 65° C. for 4 min, 95° C. for 20 s, 58° C. for 20 s. This incubation program first cools the mixture to allow the GAT5-B1-7N primer to anneal randomly along the cDNA. Ramping up to 65° C. allows Deep Vent (exo-) to catalyze second strand synthesis. Denaturation at 95° C. separates the second strand and cooling to 58° C. allows the second strand's (extension product) complementary 5′ and 3′ sequences to form a stable loop and prevent further amplification. After quasilinear amplification, a PCR amplification is performed for 17 cycles using the GAT5 primer. Following MALBAC, 0.4 μl of 50 μM outer barcode primer is added and another 5 cycles of PCR performed with OBₘ and GAT5-B1 to produce the final product. The outer barcode primer contains (starting from the 5′ end) the Read2SP sequence, which is the Illumina read 2 sequencing priming sequence, the Gₘ sequence, which is one of ‘m’ different 4-7 nucleotide cell specific barcodes separated by ≥2 Hamming distance, and the RT3 sequence, which anneals onto the MALBAC cDNA product. The addition of the outer barcode gives a total of m×n possible barcodes. This product is purified with 0.8× Amazi beads (Aline Biosciences) to remove <150 base pair primer dimers.

### Example III

### Library Preparation

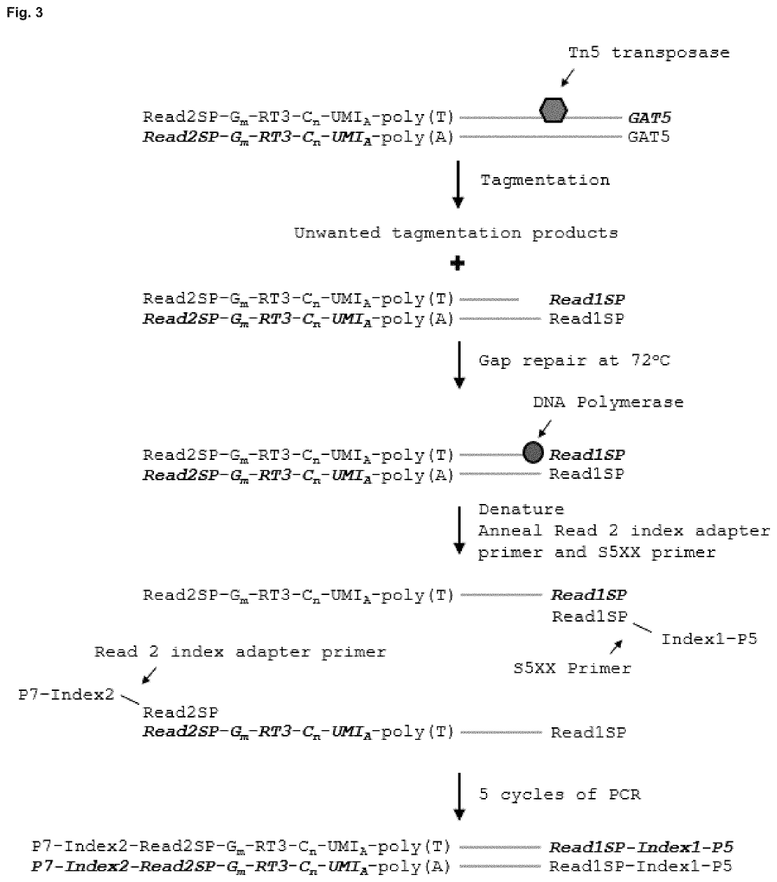

FIG. 3 illustrates a method of preparing a library for sequencing from the amplicons of Example II. The amplicon products of Example II can be prepared as an Illumina sequencing compatible library using multiple chemistries. For library preparation, a hyperactive Tn5 transposase, such as that from the Nextera DNA Library Prep Kit (Illumina), is used to attach a portion of the read 1 sequencing adapter to amplicons, then PCR is conducted with the full length sequencing adapters to produce an Illumina compatible sequencing library (FIG. 3). Tagmentation using the Nextera kit produces multiple products, with the desired product containing the barcode sequences and the read 1 sequencing priming sequence (Read1SP) flanking the cDNA. The tagmented product is added to 501 of PCR amplification mix (1× Kapa HiFi HotStart Master Mix, 0.5 μM S5XX primer (Illumina), 0.5₁M Read 2 Index Adapter primer (IDT)) and amplified using the following incubation program: 72° C. for 3 min, 98° C. for 30 s, then 5 cycles of 98° C. for 10 s, 63° C. for 30 s, and 72° C. for 3 min. The final sequencing library is purified again using 0.8× Amazi beads then sized using a Bioanalyzer (Agilent) for concentration adjustment before sequencing.

### Example IV

### Determining Tissue-Specific Transcriptional Regulatory Models within a Homogeneous Human Cell Culture

Multiple annealing and looping based amplification cycles for digital transcriptomics MALBAC-DT was performed on two human cell line as follows. The U2-OS bone osteosarcoma and HEK293T embryonic kidney cell lines were obtained from the American Type Culture Collection (ATCC. Rockville). U2-OS and HEK293T cells were maintained in Dulbecco's Modified Eagle's Medium supplemented with 10% fetal bovine serum and 100 U/ml penicillin-streptomycin (ATCC). For collection, the cells were suspended using 0.05% Trypsin-EDTA (Thermo Fisher Scientific), then washed with 1×PBS and re-suspended in Dulbecco's Modified Eagle's Medium supplemented with 10% fetal bovine serum, 2 μg/ml propidium iodide (Thermo Fisher Scientific) and 1 μM calcein AM (BD Bioscience). Live single cells with a positive calcein AM signal and negative propidium iodide signal were sorted using a MoFlo Astrios (Beckman Coulter) into 96-well plates where each well contained 3 μl of lysis buffer (1× SuperScript IV Buffer (Thermo Fisher Scientific), 0.5% IGEPAL CA-630 (Sigma-Aldrich), 500 mM dNTP, 6 mM MgSO₄, 1M Betaine, 1U SUPERase In RNase Inhibitor (Thermo Fisher Scientific), 2.5 μM ‘RT-A’ reverse transcription primer (IDT), 2.4×10⁷ dilution of ERCC's). The RT-A primer contained (starting from the 5′ end) the GAT5 sequence, which was used to create self-annealing loops during cDNA amplification, the B1 spacer sequence, the RT3 sequence, which was used as an annealing site for the outer barcode primer during the final PCR step, the C sequence, which was one of ‘n’ different 6 nucleotide cell specific barcodes separated by ≥3 Hamming distance, the UMI_{A} sequence, which was a reduced complexity random 20-mer with ˜3.5 billion (3²⁰) possible combinations to uniquely barcode each transcript, and a 12-nucleotide poly(T) tract (Table 1).

For cDNA synthesis, plates were centrifuged, incubated at 72° C. for 3 mins to denature RNA secondary structure, then cooled to 4° C. to allow primer annealing, 1 ul of reverse transcription mix (1× SuperScript IV Buffer, 0.1M DTT, 1U SUPERase In RNase Inhibitor, 60U SuperScript IV (Thermo Fisher Scientific) was added and the mixture incubated at 55° C. for 10 minutes to catalyze cDNA synthesis. To prevent excess RT-A primers from annealing during later cDNA amplification, 2 μl primer digestion mix (1× Exonuclease 1 Buffer (NEB), 12U Exonuclease I (NEB), 2.5 uM ‘RT-B’ reverse transcription primer (IDT)) was added and incubated at 37° C. for 30 minutes to digest reverse transcription primers. The RT-B primer is identical to RT-A except it contains the UMI_{B} pattern instead of the UMI_{A} pattern (Table 1), which allowed exonuclease digestion efficiency to be measured since incomplete digestion will result in cDNA amplification products with a mixture of UMI_{A} and UMI_{B} barcodes. Following digestion, the mixture was heated to 80° C. for 20 minutes to degrade the RNA and heat inactivate Exonuclease I and SuperScript IV.

The resulting cDNA was amplified using Multiple Annealing and Looping Based Amplification Cycles (MALBAC) (FIG. 2). For MALBAC, 24 μl of cDNA amplification mix (1× ThermoPol buffer (NEB), 200 μM dNTP, 1.25 mM MgSO₄, 50 μM ‘GAT5-B1-7N’ primer (IDT), 50 μM ‘GAT5-B1’ primer (IDT), 2U Deep Vent (exo-) DNA Polymerase (NEB)) was added to the cDNA synthesis mix. Quasilinear cDNA amplification was conducted by heating the mixture to 95° C. for 5 minutes then repeating 10 cycles of 4° C. for 50 s, 10° C. for 50 s, 20° C. for 50 s, 30° C. for 50 s, 40° C. for 45 s, 50° C. for 45 s, 65° C. for 4 min, 95° C. for 20 s, 58° C. for 20 s. After quasilinear amplification, a PCR amplification was performed by heating to 98° C. for 1 min then repeating the following incubation program 17 times: 95° C. for 20 s, 58° C. for 30 s, 72° C. for 3 mins. Following MALBAC, 0.41 μl of 501M outer barcode primer (see Table 1 for sequence) was added and another round of PCR performed by heating to 95° C. for Imin, repeating 5 cycles of 95° C. for 20 s, 58° C. for 30 s, and 72° C. for 3 min, then incubating at 72° C. for 5 min. The outer barcode primer contained (starting from the 5′ end) the Read2SP sequence, which was the Illumina read 2 sequencing priming sequence, the Gₘ sequence, which was one of ‘m’ different 4-7 nucleotide cell specific barcodes separated by ≥2 Hamming distance, and the RT3 sequence, which annealed onto the MALBAC cDNA product. The addition of the outer barcode gave a total of m×n possible barcodes. This product was purified with 0.8× Amazi beads (Aline Biosciences) to remove <150 base pair primer dimers.

The product was prepared as an Illumina sequencing compatible library using the Nextera DNA Library Prep Kit (Illumina). Tagmentation using the Nextera kit produced multiple products, with the desired product containing the barcode sequences and the read 1 sequencing priming sequence (Read1SP) on one side of the cDNA, and the N5XX sequence on the other. The tagmented product was added to PCR amplification mix to make 50 μl total PCR mix (1× Kapa HiFi HotStart Master Mix, 0.5 μM N5XX primer (Illumina), 0.5 μM Read 2 Index Adapter primer (IDT)) and amplified by heating to 72° C. for 3 min, 98° C. for 30 s, then repeating 5 cycles of 98° C. for 10 s, 63° C. for 30 s, and 72° C. for 3 min. The products were purified using 0.8× Amazi beads, eluted to 20 ul, then size-selected for 300-500 bp bands using an E-Gel SizeSelect 2% Agarose Gel (Fisher), then quantified using a Bioanalyzer (Agilent) for concentration adjustment before loading onto a HiSeq 4000 (Illumina) for sequencing.

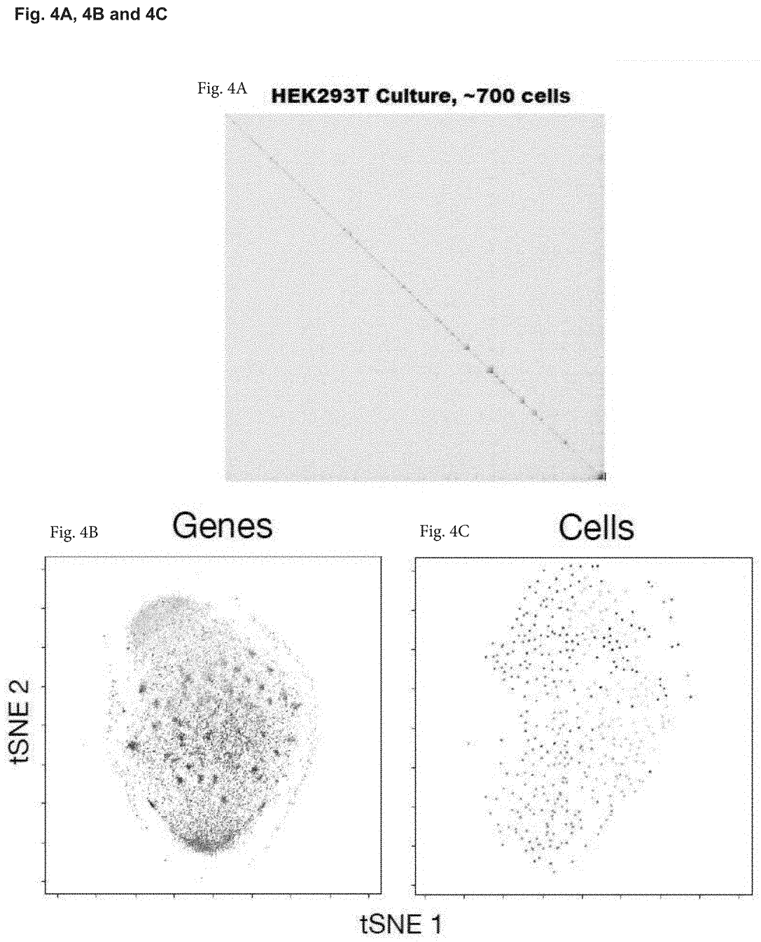

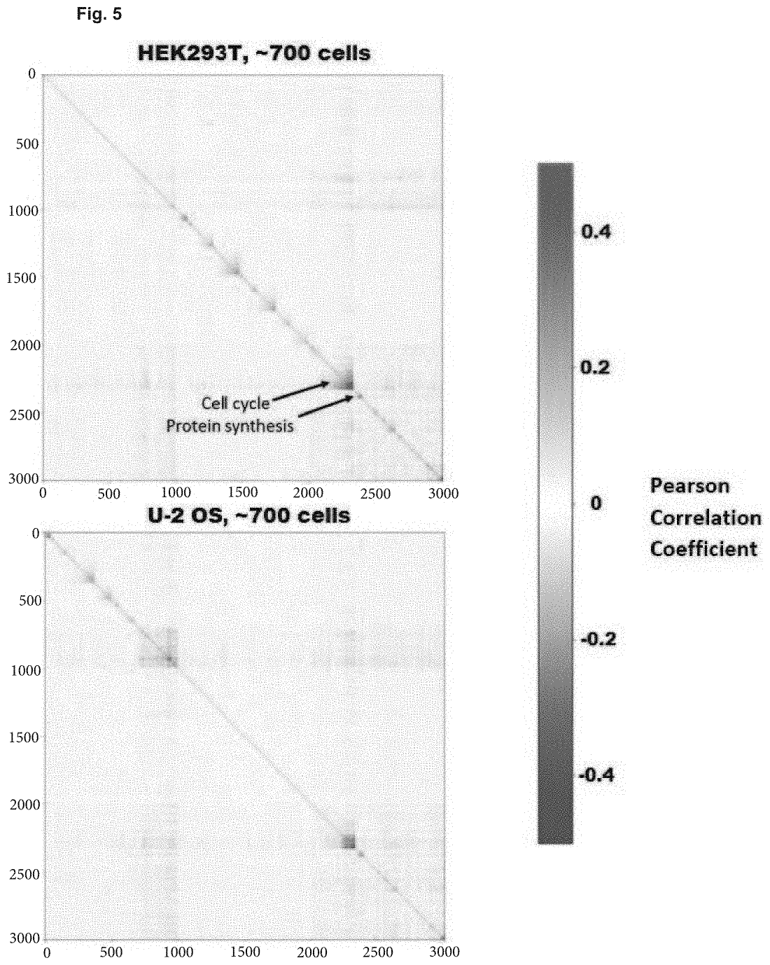

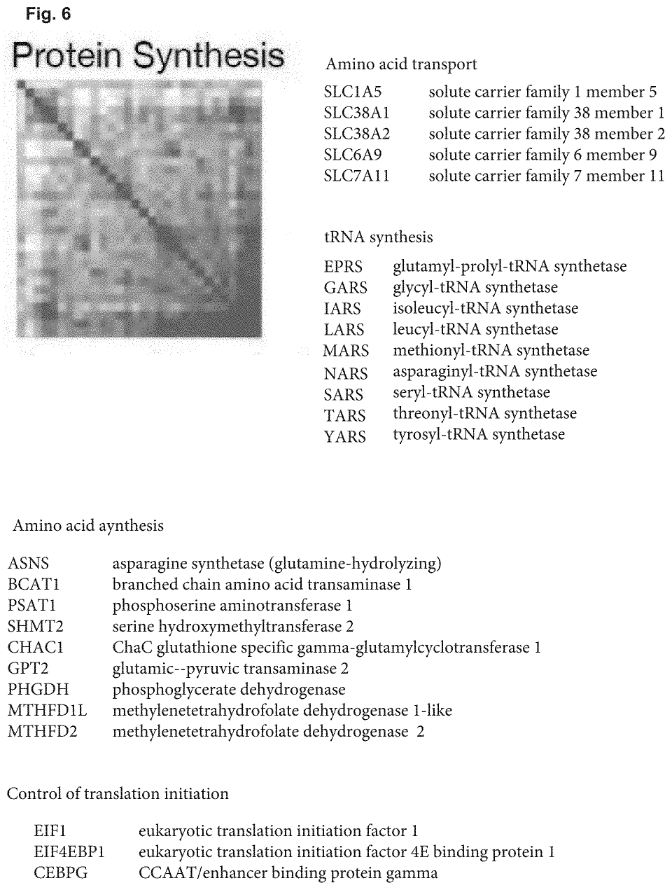

About 700 homogenously cultured HEK293T cells and about 700 homogenously cultured U-2 OS cells were sequenced with an average sequencing depth of 10⁶ reads per cell, 80% of the reads map to the exome suggesting that the library accurately reflects the transcriptome. At this depth, 12,000 genes were consistently detected. The gene expression correlation matrix for HEK293T is shown in FIG. 4A. Each square block on the diagonal indicates a gene cluster in which strong correlation is observed. These observations are from fluctuations in a culture at non-equilibrium steady state. There are total of about 100-200 clusters amongst the 12,000 genes. FIG. 4B depicts clustering of genes (left) and FIG. 4C depicts clustering of cells (right) for the HEK293T dataset using the t-stochastic neighbor embedding algorithm (t-SNE). In the gene clustering plot of FIG. 4B, each gene cluster corresponds to a square in the correlation matrix. In the gene clustering plot, each dot is one of the 12,000 genes and each cluster corresponds to a square in the correlation matrix. In the cell clustering plot of FIG. 4C, each dot is one of about 700 HEK cells, and there are no resolvable clusters. This means that the gene clusters are not a result of clusters of phenotypically different cells. A comparison of gene clusters is shown in FIG. 5 for 3000 out of 12,000 genes for HEK293T (upper). A comparison of gene clusters is shown in FIG. 5 for 3000 out of 12,000 genes for U-2 OS (lower). There are some common clusters between the two cell lines, such as those involved in cell cycle and protein synthesis. However, there are also different gene clusters which are likely cell-type specific transcriptional regulatory processes. FIG. 6 highlights the protein synthesis cluster labeled in FIG. 5. Genes in this cluster are enriched for those involved in tRNA synthesis, amino acid synthesis, amino acid transport, and control of translation initiation, all of which are important in the protein synthesis process. Therefore, correlated gene clusters have related biological functions and transcriptional regulation.

### Example V

### Kits

The materials and reagents required for the disclosed reverse transcription and amplification method may be assembled together in a kit. The kits of the present disclosure generally will include at least reverse transcriptase, and reverse transcription primers, degradation enzyme, nucleotides, DNA polymerase and extension and amplification primers described herein necessary to carry out the claimed method. In a preferred embodiment, the kit will also contain directions for reverse transcribing the RNA to cDNA and amplifying the cDNA. In each case, the kits will preferably have distinct containers for each individual reagent, enzyme or reactant. Each agent will generally be suitably aliquoted in their respective containers. The container means of the kits will generally include at least one vial or test tube. Flasks, bottles, and other container means into which the reagents are placed and aliquoted are also possible. The individual containers of the kit will preferably be maintained in close confinement for commercial sale. Suitable larger containers may include injection or blow-molded plastic containers into which the desired vials are retained. Instructions are preferably provided with the kit.

### Embodiments

The present disclosure provides a method of amplifying an RNA template strand including reverse transcribing the RNA template strand into a cDNA template strand using a reverse transcriptase and a reverse transcription primer sequence having a 3′ poly(T) sequence complementary to a 5′ poly(A) sequence of the RNA template strand, wherein the reverse transcription primer sequence further includes a 5′ self-annealing sequence, a barcode primer annealing site, a first cell specific barcode sequence having between 4 and 12 nucleotides and a first unique molecular identifier barcode sequence having between 10 to 30 nucleotides, wherein the cDNA template strand includes the reverse transcription primer sequence 5′ of the cDNA template strand and the cDNA template strand is hybridized to the RNA strand, digesting excess reverse transcription primer sequences with an enzyme, degrading the RNA strand to produce the cDNA template strand as a single strand, inactivating the reverse transcriptase, inactivating the enzyme. (a) generating a complementary strand to the cDNA template strand including the reverse transcription primer sequence using a DNA polymerase and an extension primer including the self-annealing sequence at the 5′ end of the primer, wherein the complementary strand includes the self-annealing sequence at the 5′ end and its complement at the 3′ end, (b) denaturing the cDNA template strand from the complementary strand and looping the complementary strand by annealing of the self-annealing sequence at the 3′ end and its complement at the 5′ end so as to inhibit amplification of the complementary strand, repeating steps (a) and (b) a plurality of times to generate a plurality of looped complementary strands from the cDNA template strand, denaturing the plurality of looped complementary strands and amplifying the denatured complementary strands using an amplification primer including the self-annealing sequence to produce double stranded amplicons including the reverse transcription primer sequence, denaturing the double stranded amplicons and repeatedly amplifying the denatured amplicons a plurality of times using (1) an outer barcode primer having a 3′ sequence complementary to the barcode primer annealing site, wherein the outer barcode primer further includes a 5′ self-annealing sequence, a sequencing priming sequence and a second cell specific barcode sequence having between 4 and 12 nucleotides, and (2) a primer including a 3′ self-annealing sequence to produce resulting double stranded amplicons having a first cell specific barcode sequence, a second cell specific barcode sequence and a first unique molecular identifier barcode sequence. According to one aspect, the RNA is messenger RNA, transfer RNA, ribosomal RNA, long noncoding RNA, or small interfering RNA. According to one aspect, the RNA is from a single cell. According to one aspect, the RNA is from a single cell within a heterogeneous population of cells. According to one aspect, the RNA is from a single prenatal cell. According to one aspect, the RNA is from a single cancer cell. According to one aspect, the RNA is from a single circulating tumor cell. According to one aspect, the reverse transcriptase is SuperScript II, III or IV, M-MLV Reverse Transcriptase, Maxima Reverse Transcriptase, Protoscript Reverse Reverse Transcriptase, or Thermoscript Reverse Transcriptase. According to one aspect, the 3′ poly(T) sequence includes between 10 and 30 T nucleotides. According to one aspect, the self-annealing sequence is GAT5 or GAT1. According to one aspect, the barcode primer annealing site is RT3, Read1SP or Read2SP. According to one aspect, the enzyme is a polymerase having strand displacement activity or has 5′ to 3′ exonuclease activity. According to one aspect, the enzyme is Φ29 Polymerase. Bst Polymerase, Pyrophage 3173, Vent Polymerase. Deep Vent polymerase, TOPO Taq DNA polymerase, Taq polymerase. T7 polymerase, Vent (exo-) polymerase, Deep Vent (exo-) polymerase, 9° Nm Polymerase, Klenow fragment of DNA Polymerase I, MMLV Reverse Transcriptase. AMV reverse transcriptase. HIV reverse transcriptase, a mutant form of T7 phage DNA polymerase that lacks 3′-5′ exonuclease activity, Taq polymerase. Bst DNA polymerase (full length), E. coli DNA polymerase, LongAmp Taq polymerase, OneTaq DNA polymerase, Q5. Phusion or Kapa HiFi. According to one aspect, the RNA strand is degraded at a temperature of between 75° C. and 85° C. According to one aspect, the reverse transcriptase and the enzyme are inactivated at a temperature of between 75° C. and 85° C. According to one aspect, the extension primer anneals to the cDNA template strand at a temperature of between 0° C. and 10° C. According to one aspect, the complementary strand is generated at a temperature of between 10° C. and 65° C. According to one aspect, looping the complementary strand occurs at a temperature of between 55° C. and 60° C. According to one aspect, steps (a) and (b) are repeated between 7 and 12 times. According to one aspect, amplifying the denatured complementary strands is carried out using polymerase chain reaction. According to one aspect, amplifying the denatured complementary strands is carried out using between 15 and 20 cycles of polymerase chain reaction. According to one aspect, amplifying the denatured amplicons is carried out using polymerase chain reaction. According to one aspect, the denatured amplicons are repeatedly amplified using between 3 and 7 cycles of PCR. According to one aspect, the resulting double stranded amplicons are processed for sequencing. According to one aspect, the first unique molecular identifier barcode sequence includes a semi-random sequence pattern. According to one aspect, the step of digesting excess transcription primers with an enzyme includes adding reverse transcription primers with a second unique molecular identifier barcode sequence having between 10 to 30 nucleotides includes a semi-random sequence pattern and which is different from the first unique molecular identifier barcode sequence.

What is claimed is:

1. A method of amplifying an RNA template strand comprising
reverse transcribing the RNA template strand into a cDNA template strand using a reverse transcriptase and a reverse transcription primer sequence having a 3′ poly(T) sequence complementary to a 5′ poly(A) sequence of the RNA template strand, wherein the reverse transcription primer sequence further includes a 5′ self-annealing sequence, a barcode primer annealing site, a first cell specific barcode sequence having between 4 and 12 nucleotides and a first unique molecular identifier barcode sequence having between 10 to 30 nucleotides, wherein the cDNA template strand includes the reverse transcription primer sequence 5′ of the cDNA template strand and the cDNA template strand is hybridized to the RNA strand,
digesting excess reverse transcription primer sequences with an enzyme,
degrading the RNA strand to produce the cDNA template strand as a single strand,
inactivating the reverse transcriptase,
inactivating the enzyme,
(a) generating a complementary strand to the cDNA template strand including the reverse transcription primer sequence using a DNA polymerase and an extension primer including the self-annealing sequence at the 5′ end of the primer, wherein the complementary strand includes the self-annealing sequence at the 5′ end and its complement at the 3′ end,
(b) denaturing the cDNA template strand from the complementary strand and looping the complementary strand by annealing of the self-annealing sequence at the 3′ end and its complement at the 5′ end so as to inhibit amplification of the complementary strand,
repeating steps (a) and (b) a plurality of times to generate a plurality of looped complementary strands from the cDNA template strand,
denaturing the plurality of looped complementary strands and amplifying the denatured complementary strands using an amplification primer including the self-annealing sequence to produce double stranded amplicons including the reverse transcription primer sequence,
denaturing the double stranded amplicons and repeatedly amplifying the denatured amplicons a plurality of times using (1) an outer barcode primer having a 3′ sequence complementary to the barcode primer annealing site, wherein the outer barcode primer further includes a 5′ self-annealing sequence, a sequencing priming sequence and a second cell specific barcode sequence having between 4 and 12 nucleotides, and (2) a primer including a 3′ self-annealing sequence to produce resulting double stranded amplicons having a first cell specific barcode sequence, a second cell specific barcode sequence and a first unique molecular identifier barcode sequence.

2. The method of claim 1 wherein the RNA is messenger RNA, transfer RNA, ribosomal RNA, long noncoding RNA, or small interfering RNA.

3. The method of claim 1 wherein the RNA is from a single cell.

4. The method of claim 1 wherein the RNA is from a single cell within a heterogeneous population of cells.

5. The method of claim 1 wherein the RNA is from a single prenatal cell.

6. The method of claim 1 wherein the RNA is from a single cancer cell.

7. The method of claim 1 wherein the RNA is from a single circulating tumor cell.

8. The method of claim 1 wherein the reverse transcriptase is SuperScript II, III or IV, M-MLV Reverse Transcriptase, Maxima Reverse Transcriptase, Protoscript Reverse Reverse Transcriptase, or Thermoscript Reverse Transcriptase.

9. The method of claim 1 wherein the 3′ poly(T) sequence includes between 10 and 30 T nucleotides.

10. The method of claim 1 wherein the self-annealing sequence is GAT5 or GAT1.

11. The method of claim 1 wherein the barcode primer annealing site is RT3, Read1SP or Read2SP.

12. The method of claim 1 wherein the enzyme is a polymerase having strand displacement activity or has 5′ to 3′ exonuclease activity.

13. The method of claim 1 wherein the enzyme is 029 Polymerase, Bst Polymerase, Pyrophage 3173, Vent Polymerase, Deep Vent polymerase, TOPO Taq DNA polymerase, Taq polymerase, T7 polymerase, Vent (exo-) polymerase, Deep Vent (exo-) polymerase, 9° Nm Polymerase, Klenow fragment of DNA Polymerase I, MMLV Reverse Transcriptase, AMV reverse transcriptase, HIV reverse transcriptase, a mutant form of T7 phage DNA polymerase that lacks 3′-5′ exonuclease activity, Taq polymerase, Bst DNA polymerase (full length), E. coli DNA polymerase, LongAmp Taq polymerase, OneTaq DNA polymerase, Q5, Phusion or Kapa HiFi.

14. The method of claim 1 wherein the RNA strand is degraded at a temperature of between 75° C. and 85° C.

15. The method of claim 1 wherein the reverse transcriptase and the enzyme are inactivated at a temperature of between 75° C. and 85° C.

16. The method of claim 1 wherein the extension primer anneals to the cDNA template strand at a temperature of between 0° C. and 10° C.

17. The method of claim 1 wherein the complementary strand is generated at a temperature of between 10° C. and 65° C.

18. The method of claim 1 wherein looping the complementary strand occurs at a temperature of between 55° C. and 60° C.

19. The method of claim 1 wherein steps (a) and (b) are repeated between 7 and 12 times.

20. The method of claim 1 wherein amplifying the denatured complementary strands is carried out using polymerase chain reaction.

21. The method of claim 1 wherein amplifying the denatured complementary strands is carried out using between 15 and 20 cycles of polymerase chain reaction.

22. The method of claim 1 wherein amplifying the denatured amplicons is carried out using polymerase chain reaction.

23. The method of claim 1 wherein the denatured amplicons are repeatedly amplified using between 3 and 7 cycles of PCR.

24. The method of claim 1 wherein the resulting double stranded amplicons are processed for sequencing.

25. The method of claim 1 wherein the first unique molecular identifier barcode sequence includes a semi-random sequence pattern.

26. The method of claim 1 wherein the step of digesting excess transcription primers with an enzyme includes adding reverse transcription primers with a second unique molecular identifier barcode sequence having between 10 to 30 nucleotides includes a semi-random sequence pattern and which is different from the first unique molecular identifier barcode sequence.
